(19) United States
(12) Patent Application Publication
(10) Pub. No.: US 2013/0172346 A1
(43) Pub. Date: Jul. 4, 2013

(54) USE OF KYNURENIC ACID AMIDE DERIVATIVES FOR THE TREATMENT OF HUNTINGTON'S DISEASE

(71) Applicants: László Vécsei, Szeged (HU); Dénes Zádori, Bordany (HU); Péter Klivényi, Szeged (HU); Ferenc Fülöp, Szeged (HU); István Szatmári, Szeged (HU); József Toldi, Szeged (HU); Tamás Freund, Budapest (HU); Gábor Nyiri, Budapest (HU); András Szönyi, Monor (HU)

(72) Inventors: László Vécsei, Szeged (HU); Dénes Zádori, Bordany (HU); Péter Klivényi, Szeged (HU); Ferenc Fülöp, Szeged (HU); István Szatmári, Szeged (HU); József Toldi, Szeged (HU); Tamás Freund, Budapest (HU); Gábor Nyiri, Budapest (HU); András Szönyi, Monor (HU)

(21) Appl. No.: 13/806,699

(22) PCT Filed: Jun. 29, 2011
(86) PCT No.: PCT/HU2011/000062

§ 371 Date: Feb. 8, 2013

(30) Foreign Application Priority Data
Jun. 29, 2010 (HU) P1000343

(51) Int. Cl. C07D 215/48; C07D 401/12; C07D 413/12

(52) U.S. Cl. CPC C07D 215/48; C07D 413/12; C07D 401/12
USPC 514/235.2; 546/156; 514/312; 544/128

(57) ABSTRACT

The invention is directed to kynurenic acid analogues and pharmaceutically acceptable salts thereof and pharmaceutical compositions containing said compounds for treating the symptoms of Huntington's disease and preventing the development of the symptoms.

The present invention is directed to kynurenic acid amide analogues as well as salts thereof, to pharmaceutical compositions containing said compounds for use for curing the symptoms of Huntingtons's disease as well as for preventing the development of said symptoms.

Huntington's disease is an autosomal dominantly inherited neurodegenerative clinical picture, typically occurring with people of middle age. The clinical picture is dominated by three syndromes: cognitive decline, psychopathological disturbances and motor symptoms (Walker, F. O. Huntington's Disease. Semin Neurol. 27:143-150; 2007). The pathological changes are almost exclusively restricted to the central nervous system, striatum is particularly affected. The most expressed mark is the destruction of the medium-sized spiny neurons of the striatum (Gárdián, G., Vécsei, L., Huntington's Disease: pathomechanism and therapeutic perspectives. J. Neural Transm. 111:1485-1494; 2004). Although Huntington's disease may be classified as a neurodegenerative clinical picture, it is a disease of separate entity, that can, as a whole, be unambiguously distinguished from other clinical pictures belonging to the same group due to the genetic background—which may be in 100% characterized by the autosomal dominantly inherited mutation—, the clinical picture as well as the characteristic striatal affectedness.

Although the prevalence of Huntington's Disease is not too high (˜5/100000), the clinical picture is of progressive character and leads certainly to death. Out of the motor symptoms more progressive walking problems occurring during the process in brady- and hypokinesia, are of special importance, as loosing independence in motility leads to the need of permanent medical attendance. In spite of intensive research a reliable treatment has not been solved yet, considering the complexity of the symptoms occurring during the disease. It is therefore important to make special efforts in order to develop novel therapeutically active compounds. At present there is no causal therapy, only various compounds are available, that may alleviate some symptoms occurring during the illness. (Adam, O. R., Jankovic, J. Symptomatic treatment of Huntington disease. Neurotherapeutics. 5:181-197; 2008).

For animal testing of the disease and for testing compounds of protective activity transgenic mice are most widely used, as out of the available models changes developing in these mice represent the human process relatively well and simultaneously the prolificacy of these animals ensures the testing, that can be performed with a high number of elements. One of these models is strain N171-82Q, prepared at the end of the 90-s (Schilling, G., Becher, M. W., Sharp, A. H. et al. Intranuclear inclusions and neuritic aggregates in transgenic mice expressing mutant N-terminal fragment of huntingtin. Hum. Mol. Gen. 8:397-407; 1999). The symptoms can be first observed typically at the age of about 2 months. After this age the body weight of the transgenic animals does not increase anymore, tremor, reduced motor activity, coordination disturbance and abnormal walking develop. At the end stage the body weight of the animals decreases and they die at the age of 110-130 days on the average. Surprisingly the transgenic animals do not show a great striatal cell destruction, characteristic for human cases. The reduction of the size of the neurons, indicating the existence of a severe dysfunction, however, seems to be suitable for testing the neuroprotective activity of the compounds. (Ferrante, R. J., Andreassen, O. A., Dedeoglu, A. et al. Therapeutic effects of coenzyme Q10 and remacemide in transgenic mouse models of Huntington's disease. J. Neurosci. 22:1592-1599; 2002).

Although in animal tests, by raising the endogenic kynurenic acid level (an end product of a side reaction in the triptophan metabolism), a protective activity has been achieved against the neurotoxic effect of quinolinic acid (also a triptophan metabolite) (Harris, C. A., Miranda, A. F., Tanguay, J. J. et al. Modulation of striatal quinolinate neurotoxicity by elevation of endogeneous brain kynurenic acid. Br. J. Pharmacol. 124:391-399; 1998), the systemic administration of kynurenic acid (KYNA) for the treatment of Huntingtons's disease cannot be taken into account because of the disadvantageous pharmacokinetic properties of the compound. Among the disadvantageous properties it is important to emphasize that the solubility of KYNA is poor, it can hardly pass the blood brain-barrier and it is rapidly eliminated from the brain and organism mediated by organic anion transporters. Therefore such synthetic compounds had to be elaborated, which may be more suitable for medical use due to their higher solubility, possibly better blood-brain barrier passage and more delayed elimination. The therapeutic significance of the KYNA analogues is also enhanced by the fact that if said analogues maintain the wide-spectrum receptorial effect of the KYNA, they are capable of wide spectrum anti-excitotoxic activity. KYNA is able to block the N-methyl-D-aspartate (NMDA) receptors at their strychnine insensitive glycine site and can also reduce the glutamate release by blocking the presinaptically located alpha-7-nicotinic acetylcholine receptors. Further, it has been shown, that numerous KYNA amides are capable of acting on NMDA receptors comprising NR2B sub-units, said NMDA receptors comprising these sub-units play a particularly significant role in the excitotoxicity induced by the glutamate. As out of the NMDA receptor inhibitors the glycine and polyamine site active ingredients, the NR2B sub-unit specific antagonists and channel blockers of low affinity possess an acceptable side effect profile (Muir, K. W. Glutamate-based therapeutic approaches: clinical trials with NMDA antagonists. Curr. Opin. Pharmacol. 6:53-60; 2006), the KYNA amide analogues show a considerably better side effect profile compared to the other anti-glutamatergic agents, providing a further benefit for the patients besides the good therapeutic effect.

The object of the present invention was to find suitable KYNA analogues for curing the symptoms of Huntington's disease and for preventing the development of said symptoms, as so far no such type of compounds has been investigated for this purpose.

We have now found that a specific group of KYNA amide analogues, e.g. 2-(2-N,N-dimethylaminoethylamino-1-carbonyl)-1H-quinolin-4-one hydrochloride surprisingly shows a so far unknown multiple protective effect in the N171-82Q transgenic model of Huntington's disease. We have tested the activity of the above mentioned compound for the survival, motor activity; body weight of the transgenic animals, for the size of the neurons in the striatum, i.e. the test was performed in a rather wide range from the point of view of the representation of human changes by animal tests.

The benefit of 2-(2-N,N-dimethylaminoethylamino-1-carbonyl)-1H-quinolin-4-one hydrochloride in an in vitro model of epilepsy has been disclosed (Marosi, M., Nagy, D., Farkas, T. et al. A novel kynurenic acid analogue: a comparison with kynurenic acid. An in vitro electrophysiological study. J. Neural. Transm. 117:183-188; 2010), and the term Huntington's disease was listed in the publication, but the possible activity of 2-(2-N,N-dimethylaminoethylamino-1-carbonyl)-1H-quinolin-4-one hydrochloride against Huntington's disease was not disclosed, one could not directly get to this conclusion on the basis of this publication, particularly because epilepsy and Huntington's disease are two well distinguishable diseases possessing a separate entity and the methods given in said article are completely unsuitable for model-based testing of Huntington's disease.

EP 0303 387 A1 discloses compounds very similar to KYNA amides of the general formula (1) disclosed in the present invention, but none of the disclosed particular compounds is identical with our compounds of the general formula (1). Although the term Huntington's disease is mentioned in EP 0303 387 A1, but it occurs only as an example in the enumeration of the neurodegenerative diseases. The actual pharmacological activity, however, is not supported by any experimental test results.

Considering what was said above, the surprising novelty of the invention resides in the activity of the compounds of the general formula (1) selected from KYNA amide analogues, not disclosed particularly in EP 0303 387 A1, in the indication field of Huntington's disease supported by several test data.

The present invention is directed to KYNA amide analogues of the general formula (1), pharmaceutically acceptable salts thereof and pharmaceutical compositions comprising the same for use in the treatment of the symptoms of Huntington's disease and for preventing the development of said symptoms.

KYNA amide analogues that can be used in the treatment of the symptoms of Huntington's disease and for preventing the development of said symptoms may be characterized by the following general formula (1),

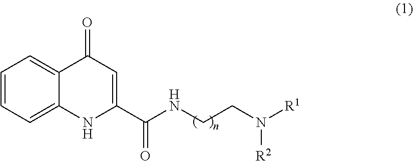

Formula (1): KYNA amide analogues,

wherein the substituents are as follows:
n stands for 1, 2, 3, 4
R¹, R² stand independently of each other for straight or branched alkyl containing 1 to 6, preferably 1 to 4 carbon atoms, preferably methyl, ethyl, n-propyl, isopropyl, n-butyl, isobutyl, sec. butyl, tert. butyl, n-pentyl, isopentyl, neopentyl, n-hexyl, particularly methyl or ethyl, or they form together with the nitrogen of the ring a 5-8 membered ring, preferably a saturated ring, comprising optionally as a further heteroatom another nitrogen or oxygen, particularly preferably a piperidinyl, pyrrolidyl, piperazyl or morpholinyl ring, wherein said rings may optionally be substituted by an alkyl containing 1 to 6 carbon atoms.

Pharmaceutically acceptable salts of the above compounds of the general formula (1) are particularly preferred for use in the treatment of the symptoms of Huntington's disease and for preventing the development of said symptoms.

The following compounds are particularly preferred for curing Huntington's disease:
2-(2-N,N-dimethylaminoethylamino-1-carbonyl)-1H-quinolin-4-one hydrochloride (2)
2-(3-N,N-dimethylpropylamino-1-carbonyl)-1H-quinolin-4-one hydrochloride (3)
2-(2-N,N-diethylaminoethylamino-1-carbonyl)-1H-quinolin-4-one hydrochloride (4)
2-(2-N-morpholino-ethylamino-1-carbonyl)-1H-quinolin-4-one hydrochloride (5)
2-(2-N-piperidylethylamino-1-carbonyl)-1H-quinolin-4-one hydrochloride (6)
2-(2-N-pyrrolidyl-ethylamino-1-carbonyl)-1H-quinolin-4-one hydrochloride (7)
2-[2-(2-N-methylpiperidyl)-methylamino-1-carbonyl]-1H-quinolin-4-one hydrochloride (8)

Synthesis for the preparation of the compounds of the general formula (1) as well as pharmaceutically acceptable salts thereof is illustrated by the following reaction scheme 1.

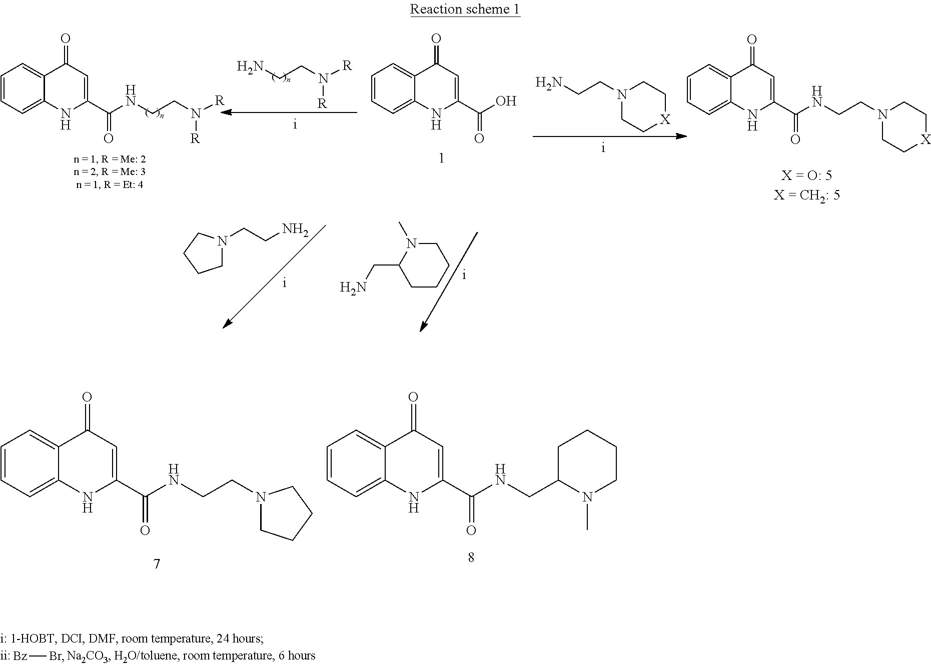

The obtained compounds are converted to their pharmaceutically acceptable acid addition salts by method known per se.

The pharmaceutically acceptable acid addition salts may be particularly suitable for pharmaceutical use due to their better solubility compared to the starting materials or the basic compounds. These acid addition salts comprise a pharmaceutically acceptable anion or cation. According to the invention, the salts appropriate for medical use are those formed by inorganic acids, e.g. hydrochloric acid, hydrobromic acid, phosphoric acid, metaphosphoric acid, nitric acid or sulfuric acid, and also salts formed by organic acids, such as acetic acid, benzosulfonic acid, benzoic acid, citric acid, ethanesulfonic acid, fumaric acid, gluconic acid, glycolic acid, isothionic acid, lactic acid, lactobionic acid, maleic acid, malic acid, methansulfonic acid, succinic acid, p-toluenesulfonic acid and tartaric acid.

### Description of the Examinations

We have examined the activity of the systemically (intraperitoneally) administered KYNA amide analogue 2-(2-N,N-dimethylaminoethylamino-1-carbonyl)-1H-quinolin-4-one hydrochloride in the N171-82Q transgenic mouse model of the Huntington's disease for the survival, motor activity, body weight of the animals and for the size of the neurons of the striatum.

### Methods

### Test Animals and Treatment Protocol

The transgenic N171-82Q mice were originally obtained from Jackson Laboratories (Bar Harbor, Me., United States), later we have propagated them in our laboratories by using B6C3F1 background strains. The progeny were genotyped by PCR technique from tail-DNA. The animals were kept in transgenic cages under standard laboratory conditions (up to 5 animals/cage), and had free access to water and food. Male and female transgenic and wild type animals respectively, were equally allotted to the individual test groups. The transgenic animals were injected intraperitoneally (5 ml/kg of body weight) every day at the same time with 2-(2-N,N-dimethylaminoethylamino-1-carbonyl)-1H-quinolin-4-one hydrochloride (at a dose of 100 mg/kg of body weight, dissolved in distilled water, pH was adjusted to 6.5 with 1N NaOH). The control transgenic and wild animals, respectively obtained a salt solution comprising 0.1 M of phosphate buffer (PBS) (5 ml/kg of body weight). The animals were inoculated in the first 5 days of the week starting from the age of 7 weeks.

### Survival Test

The transgenic animals were inoculated as disclosed above with 2-(2-N,N-dimethylamino-ethylamino-1-carbonyl)-1H-quinolin-4-one hydrochloride (n=10), and the transgenic animals serving as a control, were inoculated with 0.1 M PBS (n=10) until the animals perished. As negative control wild type animals, treated with 0.1M PBS were used (n=10).

### Open-Field Test for Examining Motor Activity

A test method almost identical with the method disclosed for the survival test was used with the exception of the number of animals (n=9, n=8, n=8 corresponding to the above group assignment). The spontaneous motor activity of the mice was tested once a week 2 hours after the given daily inoculation, always at the same day of the week. The animals stayed in the test unit (48×48×40 cm box open from above) for 5 minutes, the motor parameters were registered by the aid of Conducta 1.0 software (Experimetria Ltd., Budapest, Hungary). In order to characterize the motor activity, the parameter of the covered distance was selected and the data of the examined 9 weeks (between the age of 7 and 15 weeks) were evaluated as follows: an average of the first, second, third 3 weeks was calculated for each test group and the three test groups were compared according to the obtained average values.

### Body Weight Measurements and Immunohistochemistry by Cell Volume Estimation:

The body weight of the animals was measured once a week, always at the same day of the week and at the same time of the day, starting from the age of 7 weeks. When the animals achieved the age of 16 weeks, a transcardial perfusion was performed by a physiological saline solution subsequent to deep narcosis carried out by isoflurane (Forane®; Abbott Laboratories Hungary Ltd., Budapest, Hungary), followed by fixation with 4% paraformaldehyde. The brains were removed and after post-fixation lasting 24 hours were placed to a 0.1M phosphate buffer solution comprising 0.05% of azide (PB; pH 7.4). Blocs containing the whole striatum (7 brains were worked up in each group) were excised in the middle, so that later the cerebral hemispheres could be treated separately. By using vibratome the whole striatum was cut to slices of the thickness of 60 μm, and the slices were collected systematically randomly into 7 sample dishes for each cerebral hemisphere. After washing in 0.1M PB, the sections were incubated in a 10% then a 30% sucrose solution for the purpose of cryoprotection. Following the antigen digestion by the method of freezing and defrosting, the sections of the left brain hemisphere were repeatedly washed in 0.1 M of PB, whereafter immunostaining was used. Subsequent to shaking in a 0.05 M saline solution containing Tris-buffer (TBS; pH 7.4) the free floating sections were incubated in 1% hydrogen peroxide. After washing in 0.05 M TBS and in 2% Normal Horse Serum (Vector Laboratories Inc., Burlingname, Calif., USA) the sections were incubated in 0.05 M TBS containing anti-NeuN primary antibody (clone 60, mouse monoclonal antibody; catalogue number: MAB377; lot number: LV1616015; dilution:1:5000; Millipore, Bilerica, Mass., USA) for 35 hours at room temperature. After subsequently performed washings in 0.05M TBS the sections were incubated in a biotinylated donkey anti-mouse secondary antibody diluted in 0.05M PBS (dilution: 1:1000; Jackson ImmunoResearch Laboratories Inc., West Grove, Pa., USA) for one night at 4° C. The immunohistochemical reaction was visualized with avidin biotin (ABC) kit Vectastain® (Vector Laboratories Inc. Burlingame, Calif., USA) (dilution: 1:333, in 0.05M TBS), and in the color reaction 3′,3′-diamino-benzidine (D5637; Sigma-Aldrich Ltd. Budapest, Hungary) was used. The sections were treated with osmium tetroxide diluted in 0.1 M PB, and dehydrated in ascending alcoholic gradient and acetonitrile, whereafter they were embedded into Durcupan (ACM; Fluka, Buchs, Switzerland).

On the sequence of sections embedded into the Durcupan containing the whole striatum (n=8-9) the contours of the striatum were inscribed on the basis of the online available Allen Brain Atlas: Mouse Brain (Allen Institute for Brain Science, Seattle, Wash., USA; www.alleninstitute.org) using the 10× objective of the Zeiss Axioskop 2 (Carl Zeiss MicroImaging Ltd., Gottingen, Germany) and the software of Stereo Investigator (MBF Bioscience, Williston, Vt., USA). The volume of the striatal neurons was estimated by using 100× objective in the frame of the method Optical Fractionator (parameters: grid size: 500×500 μm, size of counting framework: 40×40 μm, thickness of the counting block: 15 μm, security zone: 4 μm, using the Nucleator local test). The systematic random selection of the neurons within the counting block was ensured by counting the first focused neuron within the counting block. On the average 87±2 neurons were counted per animal. The volume of the neurons was calculated by the Stereo Investigator software on the basis of the following formula (1), wherein r is the mean radius of a neuron:

V_{N}=(4π/3)×r³ (1)

### Statistical Analysis:

The statistical analysis of our data was performed by using Statistica software (StatSoft Inc., (2008) STATISTICA version 8.0.; www.statsoft.com). The analysis of the survival data was carried out by Kaplan-Meyer survival curves and Mantel-Cox log rank test. The normality of the data of the open-field test, the body weight measurement and the striatal cell volume measurement was first examined by Shapiro-Wilk W test. As the data of the covered distance and the body weight showed a normal distribution, in former case one-way analysis of variance and Fischer LSD post hoc test were used, whereas in latter case repeated measures analysis of variance was used for comparing the experimental groups. Data of the striatal cell volume measurement showed non-normal distribution; therefore in this case Kruskal-Wallis test was used for comparing the experimental groups. The significance level was determined at p<0.05. In case of normal distribution an average±S.E.M and in case of non-normal distribution the median and the interquartile range were used in order to depict the data

### Results

The average survival time of the N171-82Q transgenic animals was significantly (30.7%) improved from 111.3±6.6 days to 145.5±12.0 days by the intraperitoneally chronically administered (dose: 100 mg per body weight) 2-(2-N,N-dimethylaminoethylamino-1-carbonyl)-1H-quinolin-4-one hydrochloride (p=0.016, FIG. 1).

In the first 3 weeks of the behavior research the treatment with 2-(2-N,N-dimethylamino-ethylamino-1-carbonyl)-1H-quinolin-4-one hydrochloride has not improved the covered distance yet, it was 19.9% less (p=0.0046) with the transgenic animals compared to the wild-type animals. Data of the second 3 weeks show positive changes for the treated transgenic animals. This positive change has become complete by the last 3 weeks of the treatment, a significant improvement of the covered distance (p=0.0094) could be observed in the transgenic animals treated with 2-(2-N,N-dimethylaminoethylamino-1-carbonyl)-1H-quinolin-4-one hydrochloride (FIG. 2).

While a continuous increase in the body weight of the wild type animals has occurred until the age of about 14 weeks, a significant increase in the body weight of the control transgenic animals could not be observed after the age of 7 weeks. A significant increase in the body weight of the treated transgenic animals, however, was observed (p=0.00022) compared to the control transgenic animals (FIG. 3).

In the striatum of the N171-82Q transgenic animals the mean volume of the neurons (663.4 μm³, interquartile range: 605.6-681 μm³) was 14% less (p=0.0022) compared to the mean volume of the neurons in the striatum of the wild type transgenic animals (739.0 μm³, interquartile range: 734.2-802.1 μm³). The treatment with 2-(2-N,N-dimethylaminoethylamino-1-carbonyl)-1H-quinolin-4-one hydrochloride successfully prevented the atrophy of the neurons, as the size of the neurons in the striatum of the treated transgenic animals (744.5 μm³, interquartile range: 716.2-787.4 μm³) was significantly larger compared to the untreated transgenic animals (FIG. 4).

### DESCRIPTION OF THE FIGURES

FIG. 1: Kaplan-Meier survival curve of transgenic animals treated with 2-(2-N,N-dimethylaminoethylamino-1-carbonyl)-1H-quinolin-4-one hydrochloride. The treatment significantly increased (p=0.016) the survival of N171-82Q transgenic mice compared to the control groups.

FIG. 2: Effect of the treatment with 2-(2-N,N-dimethylaminoethylamino-1-carbonyl)-1H-quinolin-4-one hydrochloride on the covered distance of N171-82Q transgenic mice in the open-field test. Upon the treatment a significant improvement has been observed in progress of time.

FIG. 3: Effect of the treatment with 2-(2-N,N-dimethylaminoethylamino-1-carbonyl)-1H-quinolin-4-one hydrochloride on the body weight of N171-82Q transgenic animals. The treatment has significantly increased the body weight of the animals.

FIG. 4: Effect of the treatment with 2-(2-N,N-dimethylaminoethylamino-1-carbonyl)-1H-quinolin-4-one hydrochloride on the neuron atrophy in the striatum of N171-82Q transgenic animals. The treatment has prevented the development of atrophy.

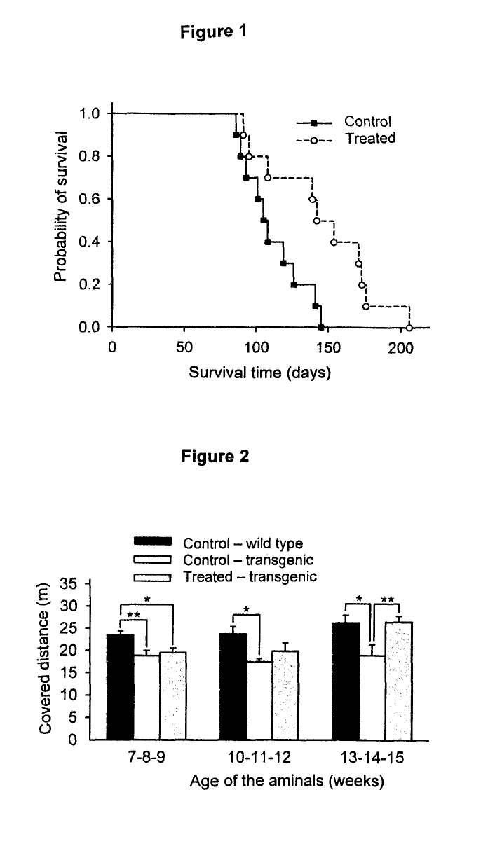

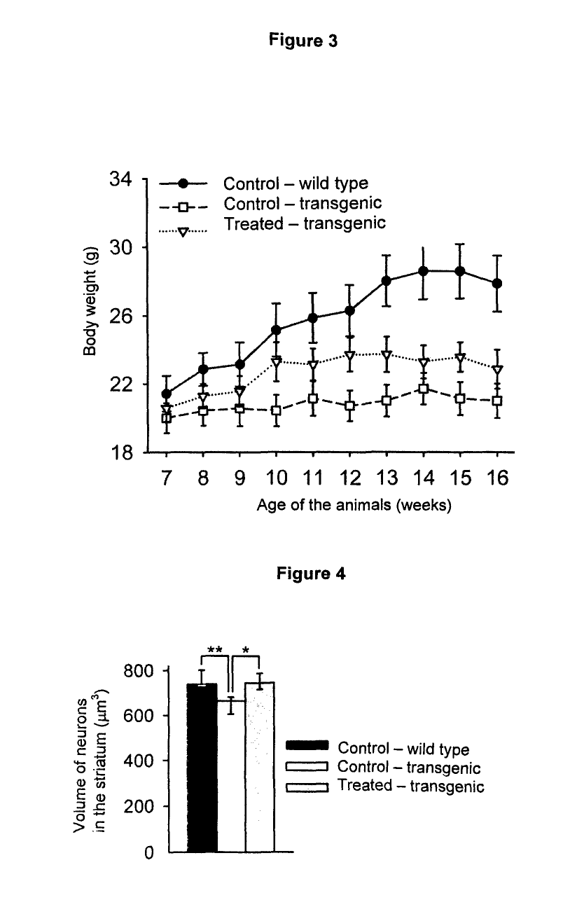

As a summary we have shown that the changes developed in the disclosed N171-82Q transgenic model of Huntington's disease could be prevented by the administration of a specific KYNA amide derivative 2-(2-N,N-dimethylaminoethylamino-1-carbonyl)-1H-quinolin-4-one hydrochloride. The use of pharmaceutical compositions comprising as active ingredient KYNA amide derivatives characterized by the general formula (1) and pharmaceutically acceptable acid addition salts thereof represents a novel therapeutic possibility in the treatment of Huntington's disease.

KYNA derivatives of the general formula (1) and their pharmaceutically acceptable acid addition salts can be administered into the organism by any possible route e.g. i.v. or orally.

The necessary daily amount (daily dose) of the KYNA amide derivatives of the general formula (1) and pharmaceutically acceptable acid addition salts thereof as an active ingredient depends on various factors, such as the route of administration and the age and status of the patient. The effective daily dosage is generally 0.01-200 mg/kg body weight, preferably 0.05 to 50 mg per/kg body weight, particularly preferably 0.05 to 20 mg/kg body weight.

It can be advantageous if the daily dosage leads to relatively constant blood concentration. This can be achieved by dividing the necessary daily dosage into two, three, four or more doses, by administering a continuous infusion of active substance for a longer period or by using continuous release formulations.

The present invention further provides pharmaceutical compositions for use in the treatment of Huntington's disease, comprising as active ingredient a pharmaceutically effective amount of KYNA amide derivatives of the general formula (1) or a pharmaceutically acceptable acid addition salt thereof together pharmaceutically acceptable carriers.

The present invention further provides pharmaceutical compositions for use in the treatment of Huntington's disease, comprising as active ingredient at least one compound of the following compounds or a combination thereof:
2-(2-N,N-dimethylaminoethylamino-1-carbonyl)-1H-quinolin-4-one hydrochloride (2)
2-(3-N,N-dimethylpropylamino-1-carbonyl)-1H-quinolin-4-one hydrochloride (3)
2-(2-N,N-diethylaminoethylamino-1-carbonyl)-1H-quinolin-4-one hydrochloride (4)
2-(2-N-morpholino-ethylamino-1-carbonyl)-1H-quinolin-4-one hydrochloride (5)
2-(2-N-piperidylethylamino-1-carbonyl)-1H-quinolin-4-one hydrochloride (6)
2-(2-N-pyrrolidyl-ethylamino-1-carbonyl)-1H-quinolin-4-one hydrochloride (7)
2-[2-(2-N-methylpiperidyl)-methylamino-1-carbonyl]-1H-quinolin-4-one hydrochloride (8)

The pharmaceutical compositions are active in Huntington's disease.

The pharmaceutical compositions may be prepared by admixing KYNA amide derivatives of the general formula (1) or pharmaceutically acceptable acid addition salts thereof with pharmaceutically acceptable carriers or other excipients.

The pharmaceutical compositions may be suitable for oral, rectal, nasal, topical (e.g. transdermal, buccal, sublingual), vaginal, parenteral (e.g. subcutaneous, intramuscular, intravenous or intradermal) administration.

The products may preferably be prepared in the form of dosage units by the conventionally used methods of drug production. During the production the active ingredient is admixed with a vehicle containing one or more supplementary components. For manufacture of the products, the active substance will usually be mixed thoroughly and evenly with the fluid vehicle or a mixture thereof, and thereafter the mixture may be further formulated, if desired.

The various routes of administration mentioned above, may exhibit various individual advantages. For example, the pharmaceutical compositions for oral administration may be prepared in physically separated units comprising a predetermined defined amount of the active ingredient, e.g. tablets, capsules, wafer products, powders or granulates; aqueous or non-aqueous (e.g. alcohol) solutions or suspensions; or in the form of oil-in-water or water-in-oil type fluid emulsions. Tablets can be produced by using optionally one or more vehicles, by compressing or molding. Compressed tablets may be prepared by methods known per se e.g. a free-flowing active ingredient in powder or granulated form, may be compressed optionally with an excipient (povidone, gelatine or hydroxypropylmethyl cellulose), lubricants, inert diluents, preservatives, disintegrating agents (e.g. sodium starch-glycolate or crospovidone), surfactants or dispersing agents, by means of an appropriate apparatus. Molded tablets are produced by molding powdered agents wetted with inert, liquid diluents into an appropriate shape. Tablets can, if necessary, be provided with a coating or pattern, and converted into forms ensuring the sustained or controlled release of the active agent with the desired release profile, e.g. by admixing hydroxypropylmethyl cellulose in varying proportions. Optionally, the tablets may be coated with an enteric coating, ensuring that the active ingredient is not released in the stomach, but in other parts of the gastro-intestinal tract.

Forms of products suitable for parenteral administration may contain antioxidants, buffers, bacteriostatic agents, and an aqueous or non-aqueous, isotonic sterile solution for injection which makes the product isotonic with the recipient's blood; or an aqueous or non-aqueous isotonic sterile injection solution, or an aqueous or non-aqueous sterile suspension, optionally comprising suspending and thickening agents, e.g. liposomes or other microparticle systems, for delivery of the active agent to the blood components or to one or more organs.

Products can be presented in the form of sealed containers, e.g. ampoules or tubes including a unit or multiple dose, or stored in a lyophilized phase, to which it is sufficient to add the appropriate sterile liquid vehicle e.g. water for injection before use. Ready-to-use solutions and suspensions for injections can be produced from sterile powders, granules and the tablets described above.

Advantageous unit-dosage products may contain the above-described daily dose or unit, the daily divided dose, or an appropriate fraction of that.

Therapeutic products covered by the invention naturally contain, in addition to the vehicles mentioned above, other vehicles conventionally used in pharmaceutical production, depending on the form of the product in question, e.g. an oral dosage form may further contain sweetening agents, thickening agents and aromatic agents.

Further details of the preparation of the active ingredients effective in the treatment of Huntington's disease are illustrated by the following examples serving merely as illustration and are not intended to restrict the scope of claims.

### Example 1

### 2-(2-N,N-dimethylaminoethylamino-1-carbonyl)-1H-quinolin-4-one hydrochloride (2)

0.40 g (2.13 mmol) of kynurenic acid are dissolved in 30 ml of dimethylformamide (DMF), 0.28 g (2.13 mmol) of 1-hydroxy benztriazole (1-HOBT) and 0.19 g (2.13 mmol) of N,N-dimethyl-ethylene diamine are added. The mixture thus obtained is stirred at 0° C. for 30 minutes whereafter 0.4 ml (2.34 mmol) of N,N′-diisopropyl carbodiimide (DCI) are added. The mixture is allowed to warm up to room temperature and it is further stirred for 24 hours. After removing the solvent, the product is purified by column chromatography, using methanol as eluent.

After evaporation the obtained free base is converted to a salt by using hydrochloric acid—ethanol—Et₂O.

0.54 g (86%); M.p.: 178-179° C. ¹H NMR (D₂O); 3.00 (6H, s, N—CH₃); 3.47 (2H, t, J=5.8 Hz, CH₂); 3.87 (2H, t, J=5.7 Hz, CH₂); 6.90 (1H, s, C3-H); 7.58 (1H, t, J=7.2 Hz, Ar—H); 7.81-7.86 (2H, m, Ar—H); 8.18 (1H, d, J=8.1 Hz, Ar—H).

### Example 2

### 2-(3-N,N-dimethylpropylamino-1-carbonyl)-1H-quinolin-4-one hydrochloride (3)

We proceed as disclosed in Example 1 using 0.22 g (2.13 mmol) of 3-dimethlyamino-1-propylamine.

0.51 g (77%), M.p.: 149-150° C. ¹H NMR (D₂O); 2.10 (2H, t, J=8.2 Hz, CH₂); 2.92 (6H, s, N—CH₃); 3.25 (2H, t, J=7.9 Hz, CH₂); 3.55 (2H, t, J=6.7 Hz, CH₂); 6.87 (1H, s, C3-H); 7.57 (1H, t, J=7.4 Hz, Ar—H); 7.83-7.86 (2H, m, Ar—H); 8.18 (1H, d, J=8.2 Hz, Ar—H).

### Example 3

### 2-(2-N,N-diethylaminoethylamino-1-carbonyl)-1H-quinolin-4-one hydrochloride (4)

We proceed as disclosed in Example 1 using 0.25 g (2.13 mmol) of N,N-diethylene diamine.

0.55 g (80%). M.p.: 192-194° C. ¹H NMR (D₂O); 1.34 (6H, t, J=7.3 Hz, N—CH₂—CH₃); 3.30-3.38 (4H, m, N—CH₂—CH₃); 3.47 (2H, t, J=6.4 Hz, CH₂); 3.86 (2H, t, J=6.4 Hz, CH₂); 6.89 (1H, s, C3-H); 7.57 (1H, t, J=7.6 Hz, Ar—H); 7.59-7.87 (2H, m, Ar—H); 8.16 (1H, d, J=8.3 Hz, Ar—H).

### Example 4

### 2-(2-N-morpholinylethylamino-1-carbonyl)-1H-quinolin-4-one hydrochloride (5)

We proceed as disclosed in Example 1 using 0.27 g (2.13 mmol) of 2-morpholinoethyl amine.

0.54 g (75%), M.p.: 230-232° C. ¹H NMR (D₂O); 3.30 (2H, bs, CH₂), 3.51 (2H, t, J=6.12 Hz, CH₂); 3.67 (2H, bs, CH₂); 3.90 (2H, t, J=5.9 Hz, CH₂); 3.91 (2H, bs, CH₂); 4.13 (2H, bs, CH₂); 6.89 (1H, s, C3-H); 7.57 (1H, t, J=7.4 Hz, Ar—H); 7.86 (1H, d, J=8.4 Hz, Ar—H); 8.19 (1H, t, J=7.1 Hz, Ar—H).

### Example 5

### 2-(2-N-piperidylethylamino-1-carbonyl)-1H-quinolin-4-one hydrochloride (6)

We proceed as disclosed in Example 1 using 0.27 g (2.13 mmol) of 1-(2-aminoethyl)-piperidine.

0.58 g (81%). M.p.: 206-208° C. ¹H NMR (D₂O); 1.48-1.99 (6H, m), 3.02 (2H, t, J=12.5 Hz, CH₂); 3.41 (2H, t, J=6.2 Hz, CH₂); 3.63-3.67 (2H, m, CH₂); 3.86 (2H, t, J=6.2 Hz, CH₂); 6.88 (1H, s, C3-H); 7.57 (1H, t, J=7.4 Hz, Ar—H); 7.81-7.89 (2H, m, Ar—H); 8.19 (1H, d, J=8.2 Hz, Ar—H).

### Example 6

### 2-(2-N-pirrolidylethylamino-1-carbonyl)-1H-quinolin-4-one hydrochloride (7)

We proceed as disclosed in Example 1 using 0.24 g (2.13 mmol) of 1-(2-aminoethyl)-pyrrolidine.

0.56 g (82%). M.p.: 185-187° C. ¹H NMR (D₂O); 2.02-2.19 (4H, m), 3.15-3.20 (2H, m CH₂); 3.52 (2H, t, J=6.0 Hz, CH₂); 3.74-3.77 (2H, m, CH₂); 3.85 (2H, t, J=6.0 Hz, CH₂); 6.89 (1H, s, C3-H); 7.56 (1H, t, J=7.4 Hz, Ar—H); 7.79-7.7.86 (2H, m, Ar—H); 8.17 (1H, d, J=8.2 Hz, Ar—H).

### Example 7

### 2-[2-(2-N-methylpiperidyl)-methylamino-1-carbonyl]-1H-quinolin-4-one hydrochloride (8)

We proceed as disclosed in Example 1 using 0.27 g (2.13 mmol) of 2-N-methyl-piperidyl-methylamine.

0.55 g (77%); M.p.: 218-220° C. ¹H NMR (D₂O); 1.55-2.32 (6H, m), 3.02 (3H, s); 3.11-3.95 (5H, m); 6.85 (1H, s, C3-H); 7.53 (1H, t, J=7.4 Hz, Ar—H); 7.77-7.82 (2H, m); 8.17 (1H, d, J=8.2 Hz, Ar—H).

1. A KYNA amide analog compound of the general formula (1), 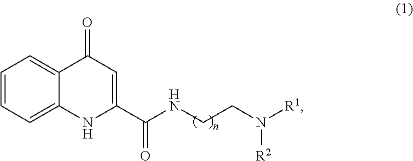
wherein the substituents are as follows:
n stands for 1, 2, 3, or 4
R¹, R² stand independently of each other for straight or branched alkyl containing 1 to 6 carbon atoms, or they form together with the nitrogen of the ring a 5-8 membered ring, comprising optionally as a further heteroatom another nitrogen or oxygen, wherein said ring may optionally be substituted by an alkyl containing 1 to 6 carbon atoms
and pharmaceutically acceptable acid addition salts thereof,
for use in the treatment of the symptoms of Huntington's disease and for preventing the development of said symptoms.

2. The compound as claimed in claim 1 selected from the group consisting of:
2-(2-N,N-dimethylaminoethylamino-1-carbonyl)-1H-quinolin-4-one hydrochloride,
2-(3-N,N-dimethylaminopropylamino-1-carbonyl)-1H-quinolin-4-one hydrochloride,
2-(2-N,N-diethylaminoethylamino-1-carbonyl)-1H-quinolin-4-one hydrochloride,
2-(2-N-morpholino-ethylamino-1-carbonyl)-1H-quinolin-4-one hydrochloride,
2-(2-N-piperidylethylamino-1-carbonyl)-1H-quinolin-4-one hydrochloride,
2-(2-N-pyrrolidyl-ethylamino-1-carbonyl)-1H-quinolin-4-one hydrochloride and
2-[2-(2-N-methylpiperidyl)-methylamino-1-carbonyl]-1H-quinolin-4-one hydrochloride,
for use in the treatment of the symptoms of Huntington's disease and for preventing the development of said symptoms.

3. 2-(2-N,N-dimethylaminoethylamino-1-carbonyl)-1H-quinolin-4-one hydrochloride as claimed in claim 1 for use in the treatment of the symptoms of Huntington's disease and for preventing the development of said symptoms.

4. Pharmaceutical composition comprising as active ingredient a pharmaceutically efficient amount of KYNA amide derivatives of the general formula (1) or pharmaceutically acceptable acid addition salts thereof, wherein the substituents are defined in claim 1, together with pharmaceutically acceptable carriers and excipients, for use in the treatment of Huntington's disease.

5. The pharmaceutical composition as claimed in claim 4, comprising as active ingredient any of the following compounds or a combination thereof:
2-(2-N,N-dimethylaminoethylamino-1-carbonyl)-1H-quinolin-4-one hydrochloride,
2-(3-N,N-dimethylaminopropylamino-1-carbonyl)-1H-quinolin-4-one hydrochloride,
2-(2-N,N-diethylaminoethylamino-1-carbonyl)-1H-quinolin-4-one hydrochloride,
2-(2-N-morpholino-ethylamino-1-carbonyl)-1H-quinolin-4-one hydrochloride,
2-(2-N-piperidylethylamino-1-carbonyl)-1H-quinolin-4-one hydrochloride,
2-(2-N-pyrrolidyl-ethylamino-1-carbonyl)-1H-quinolin-4-one hydrochloride and
2-[2-(2-N-methylpiperidyl)-methylamino-1-carbonyl]-1H-quinolin-4-one hydrochloride,
for use in the treatment of the symptoms of Huntington's disease and for preventing the development of said symptoms.

6. The pharmaceutical composition as claimed in claim 4, comprising as active ingredient 2-(2-N,N-dimethylaminoethylamino-1-carbonyl)-1H-quinolin-4-one hydrochloride, for use in the treatment of Huntington's disease.

7. The compound of claim 1, wherein R¹, R² stand independently of each other for straight or branched alkyl containing 1 to 4 carbon atoms.

8. The compound of claim 1, wherein R¹, R² stand independently of each other for methyl, ethyl, n-propyl, isopropyl, n-butyl, isobutyl, sec. butyl, tert. butyl, n-pentyl, isopentyl, neopentyl, n-hexyl, particularly methyl or ethyl.

9. The compound of claim 1, wherein R¹, R² stand independently of each other for methyl or ethyl.

10. The compound of claim 1, wherein R¹, R² form together with the nitrogen of the ring a 5-8 membered saturated ring, wherein said ring may optionally be substituted by an alkyl containing 1 to 6 carbon atoms.

11. The compound of claim 1, wherein R¹, R² form together with the nitrogen of the ring a piperidyl, pyrrolidyl, piperazyl or morpholinyl ring, wherein said ring may optionally be substituted by an alkyl containing 1 to 6 carbon atoms.

12. A method for the treatment of the symptoms of Huntington's disease or for preventing the development of said symptoms, said method comprising administering the pharmaceutical composition of claim 4 to a patient in need thereof.
